(19) United States
(12) Patent Application Publication
(10) Pub. No.: US 2020/0305727 A1
(43) Pub. Date: Oct. 1, 2020

(54) IMAGE PROCESSING DEVICE, IMAGE PROCESSING METHOD, AND PROGRAM

(71) Applicant: CANON KABUSHIKI KAISHA, Tokyo (JP)

(72) Inventor: Naoya Iizuka, Yokohama-shi (JP)

(21) Appl. No.: 16/826,902

(22) Filed: Mar. 23, 2020

(30) Foreign Application Priority Data
Mar. 28, 2019 (JP) 2019-064696

(51) Int. Cl. A61B 5/02; A61B 5/00; A61B 5/145

(52) U.S. Cl. CPC A61B 5/02007; A61B 5/0095; A61B 5/7435; G01N 29/2418; A61B 5/14546; A61B 5/7282; A61B 5/14542

(57) ABSTRACT

An image processing device disclosed in the present specification includes an acquiring unit, a determination unit, and a generation unit. The acquiring unit acquires composition image data by synthesizing at least two or more of photoacoustic image data that include volume data derived from a photoacoustic wave generated by light irradiation to a subject. The determination unit determines a generation condition of moving image data based on the composition image data. The generation unit generates the moving image data from the composition image data based on the generation condition.

### BACKGROUND

### Field

The disclosure of the present specification relates to an image processing device, an image processing method, and a program. Description of the Related Art

### Description of the Related Art

A photoacoustic apparatus that acquires characteristic information in a subject by detecting a photoacoustic wave generated by irradiating the subject with light is known. Japanese Patent Laid-Open No. 2014-68701 describes that three-dimensional volume data is generated on the basis of a photoacoustic signal that is a reception signal of a photoacoustic wave.

### SUMMARY

However, when making a moving image using a plurality of three-dimensional volume data acquired in time series, there is a problem that the observation cross section direction, image range, and time resolution of the generated moving image are not necessarily suitable for observation.

The present disclosure has been made in view of the above problem, and an aspect of the present disclosure is to generate a moving image suitable for observation.

Moreover, in addition to the aspect described above, it is also possible to position, as one of the other aspects of the present disclosure, achieving operations and effects derived from more than one configuration presented in the embodiments described below for carrying out the present disclosure, such as operations and effects that cannot be obtained by conventional techniques.

According to another aspect of the present disclosure, an image processing device disclosed in the present specification includes a first acquiring unit, a second acquiring unit, a determination unit, and a generation unit. The first acquiring unit is configured to acquire photoacoustic image data that is volume data derived from a photoacoustic wave generated by light irradiation to a subject. The second acquiring unit is configured to acquire composition image data by synthesizing at least two or more of the photoacoustic image data. The determination unit is configured to determine a generation condition of moving image data based on the composition image data. The generation unit is configured to generate the moving image data from the composition image data based on the generation condition.

Further features of the present disclosure will become apparent from the following description of exemplary embodiments (with reference to the attached drawings).

According to an aspect of the disclosure of the presentspecification, moving image data suitable for observation can be generated.

### BRIEF DESCRIPTION OF THE DRAWINGS

FIG. 1 is a block diagram illustrating an example of the configuration of a system according to the present embodiment.

FIG. 2 is a block diagram illustrating an example of an image processing device according to the present embodiment and its peripheral configuration.

FIG. 3 is a block diagram illustrating an example of a detailed configuration of a photoacoustic apparatus according to the present embodiment.

FIG. 4 is a schematic view illustrating an example of a probe according to the present embodiment.

FIG. 5 is a flowchart presenting an example of an image processing method executed by the photoacoustic apparatus according to a first embodiment.

FIG. 6 is a diagram illustrating an example of a data flow according to the first embodiment.

FIG. 7 is a flowchart presenting an example of an image processing method executed by the photoacoustic apparatus according to a second embodiment.

### DESCRIPTION OF THE EMBODIMENTS

Preferred embodiments of the image processing device disclosed in the present specification will now be described with reference to the drawings. However, the dimensions, materials, shapes, relative arrangement, and the like of the components described below should be appropriately changed according to the configuration and various conditions of the device to which the invention is applied. Therefore, the scope of the present disclosure is not intended to be limited to the following description.

A photoacoustic image obtained by the system according to the image processing device disclosed in the present specification reflects the absorbing quantity and absorption ratio of light energy. The photoacoustic image is an image representing a spatial distribution of at least one piece of subject information such as the generated sound pressure (initial sound pressure) of a photoacoustic wave, the optical absorption energy density, and the optical absorption coefficient. The photoacoustic image may be an image representing a two-dimensional spatial distribution or an image (volume data) representing a three-dimensional spatial distribution. The photoacoustic image may be an image representing a two-dimensional spatial distribution or an image representing a three-dimensional spatial distribution in the depth direction from the subject surface.

The system according to the image processing device disclosed in the present specification can generate a functional image of the subject using a plurality of photoacoustic images corresponding to a plurality of wavelengths. The functional image may be an image indicating information corresponding to the concentration of a substance constituting the subject, such as glucose concentration, collagen concentration, melanin concentration, and volume fraction of fat or water. The functional image may be an oxygen saturation image SO₂(r) generated using an absorption coefficient image μₐ^{λ}₁(r) based on a photoacoustic wave generated by light irradiation with a first wavelength λ₁ and an absorption coefficient image μₐ^{λ}₂(r) based on a photoacoustic wave generated by light irradiation with a second wavelength λ₂. For example, the system according to the present disclosure may generate an oxygen saturation image SO₂(r) as a functional image according to equation (1).

$$\begin{matrix}[ Math . 1 ] & \\ {SO}_{2} ( r ) = \frac{\frac{{μ}_{a}^{{λ}_{2}} ( r )}{{μ}_{a}^{{λ}_{1}} ( r )} {ɛ}_{Hb}^{{λ}_{1}} - {ɛ}_{Hb}^{{λ}_{2}}}{( {ɛ}_{HbO}^{{λ}_{2}} - {ɛ}_{Hb}^{{λ}_{2}} ) - \frac{{μ}_{a}^{{λ}_{2}} ( r )}{{μ}_{a}^{{λ}_{1}} ( r )} ( {ɛ}_{HbO}^{{λ}_{1}} - {ɛ}_{Hb}^{{λ}_{1}} )} & Equation ( 1 )\end{matrix}$$

Here, ε_{Hb}^{λ}₁ is a molar absorption coefficient [mm⁻¹ mol⁻¹] of deoxyhemoglobin corresponding to the first wavelength λ₁, and ε_{Hb}^{λ}₂ is a molar absorption coefficient [mm⁻¹ mol⁻¹] of deoxyhemoglobin corresponding to the second wavelength λ₂. ε_{HbO}^{λ}₁ is a molar absorption coefficient [mm⁻¹ mol⁻¹] of oxyhemoglobin corresponding to the first wavelength λ₁, and ε_{HbO}^{λ}₂ is a molar absorption coefficient [mm⁻¹ mol⁻¹] of oxyhemoglobin corresponding to the second wavelength r is a position.

Furthermore, in the system according to the image processing device disclosed in the present specification, an image indicating a ratio of a first photoacoustic image based on a photoacoustic wave generated by light irradiation with the first wavelength λ1 and a second photoacoustic image based on a photoacoustic wave generated by light irradiation with the second wavelength λ2 may be used as a functional image. That is, an image based on the ratio of the first photoacoustic image based on the photoacoustic wave generated by light irradiation with the first wavelength λ1 and the second photoacoustic image based on the photoacoustic wave generated by light irradiation with the second wavelength λ2 may be used as a functional image. Since an image generated according to the deformed equation of the equation (1) can also be expressed by the ratio between the first photoacoustic image and the second photoacoustic image, it can be said to be an image (functional image) based on the ratio between the first photoacoustic image and the second photoacoustic image.

The functional image may be an image representing a two-dimensional spatial distribution or an image representing a three-dimensional spatial distribution in the depth direction from the subject surface.

### First Embodiment

The configuration of the system and the image processing method of the present embodiment will be described below.

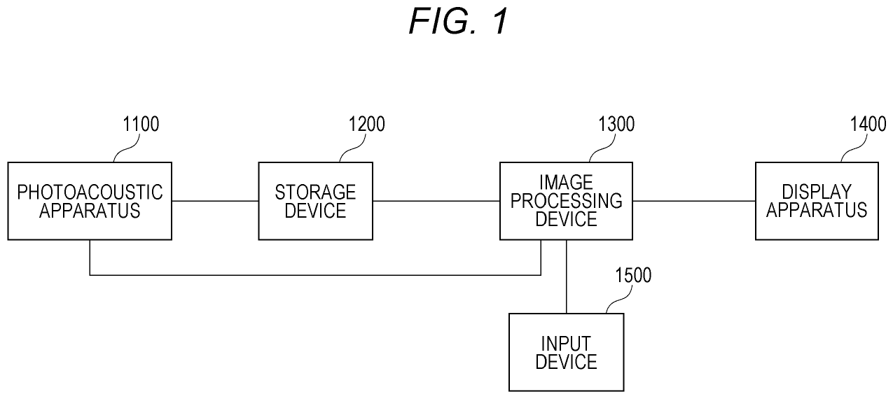

A system according to the present embodiment will be described with reference to FIG. 1. FIG. 1 is a block diagram illustrating an example of the configuration of the system according to the present embodiment. The system according to the present embodiment includes a photoacoustic apparatus 1100, a storage device 1200, an image processing device 1300, a display device 1400, and an input device 1500. Transmission and reception of data between the devices may be performed by wire or wirelessly.

The photoacoustic apparatus 1100 generates a photoacoustic image by photographing a subject 100 and outputs the image to the storage device 1200 or the image processing device 1300. The photoacoustic apparatus 1100 is an apparatus that acquires information regarding a characteristic value corresponding to each of a plurality of positions in the subject 100 using a reception signal obtained by receiving a photoacoustic wave generated by light irradiation. That is, the photoacoustic apparatus 1100 is a device that acquires image data (photoacoustic image) in which functional information related to the optical characteristics of the subject is visualized on the basis of a photoacoustic signal obtained by receiving a photoacoustic wave generated in the subject by light irradiation to the subject 100.

The storage device 1200 may be a computer readable storage medium such as a read only memory (ROM), a magnetic disk, or a flash memory. The storage device 1200 may be a storage server via a network such as a picture archiving and communication system (PACS). That is, the storage device 1200 corresponds to an example of a storage unit.

The image processing device 1300 is a device that processes a photoacoustic image and information such as supplementary information of the photoacoustic image stored in the storage device 1200.

The unit carrying the arithmetic function of the image processing device 1300 can be constituted by a processor such as a CPU or a graphics processing unit (GPU) or an arithmetic circuit such as a field programmable gate array (FPGA) chip. These units may be constituted of a plurality of processors or arithmetic circuits as well as a single processor or arithmetic circuit.

The unit carrying the storage function of the image processing device 1300 can be constituted by a non-temporary computer readable storage medium such as a read only memory (ROM), a magnetic disk, or a flash memory. The unit carrying the storage function may be a volatile medium such as a random access memory (RAM). The computer readable storage medium in which a program is stored is a non-temporary computer readable storage medium. The unit carrying the storage function may be constituted of a plurality of computer readable storage media as well as a single computer readable storage medium. That is, the unit carrying the storage function of the image processing device 1300 corresponds to an example of a storage unit.

The unit carrying the control function of the image processing device 1300 is constituted by an arithmetic element such as a CPU. The unit carrying the control function controls the operation of each component of the system. The unit carrying the control function may control each component of the system upon receiving an instruction signal by various operations such as the start of measurement from an input section 170. The unit carrying the control function may read out a program code stored in the storage unit and control the operation of each component of the system.

The display device 1400 is a display such as a liquid crystal display or an organic electro luminescence (EL). The display device 1400 may display an image and a GUI for operating the device.

As the input device 1500, an operation console constituted by a user-operable mouse, keyboard, and the like can be adopted. The display device 1400 may be constituted by a touchscreen, and the display device 1400 may be used as the input device 1500.

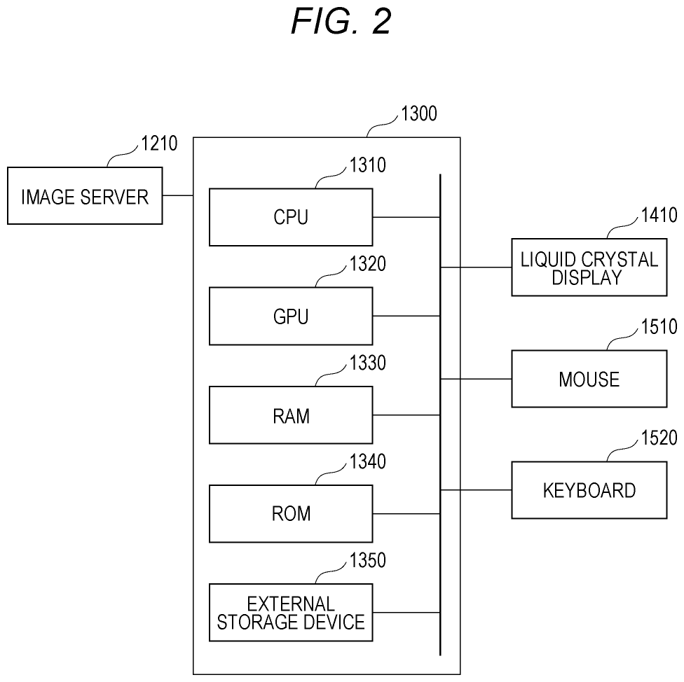

FIG. 2 illustrates a specific configuration example of the system according to the present embodiment illustrated in FIG. 1. The image processing device 1300 includes a CPU 1310, a GPU 1320, a RAM 1330, a ROM 1340, and an external storage device 1350. A liquid crystal display 1410 as the display device 1400 is connected to the image processing device 1300. Furthermore, a mouse 1510 and a keyboard 1520 as the input devices 1500 are connected. In addition, the image processing device 1300 is connected with an image server 1210 as the storage device 1200 such as a picture archiving and communication system (PACS). Due to this, image data can be stored on the image server 1210, and image data on the image server 1210 can be displayed on the liquid crystal display 1410.

Next, a configuration example of the device included in the system according to the present embodiment will be described.

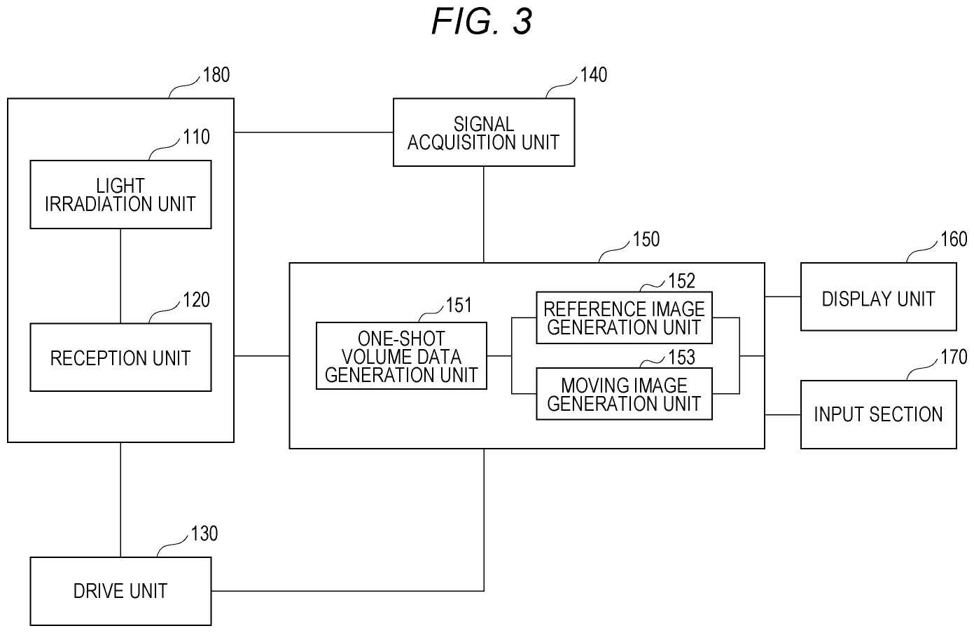

FIG. 3 is a schematic block diagram of a device included in the system according to the present embodiment.

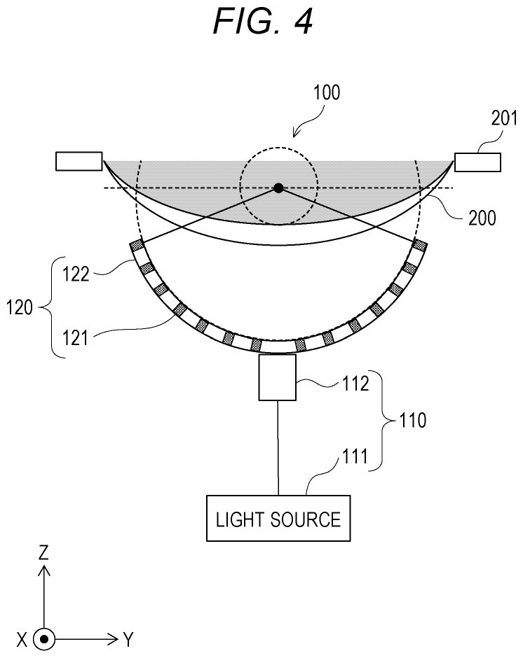

The photoacoustic apparatus 1100 according to the present embodiment includes a drive unit 130, a signal acquisition unit 140, a computer 150, and a probe 180. The probe 180 has a light irradiation unit 110 and a reception unit 120. FIG. 4 presents a schematic view of the probe 180 according to the present embodiment. The measurement object is the subject 100. The drive unit 130 drives the light irradiation unit 110 and the reception unit 120 and performs mechanical scanning. The light irradiation unit 110 irradiates the subject 100 with light, and an acoustic wave is generated in the subject 100. An acoustic wave generated by a photoacoustic effect due to light is also referred to as a photoacoustic wave. By receiving a photoacoustic wave, the reception unit 120 outputs an electric signal (photoacoustic signal) as an analog signal.

The signal acquisition unit 140 converts an analog signal having been output from the reception unit 120 into a digital signal and outputs the digital signal to the computer 150. The computer 150 stores the digital signal having been output from the signal acquisition unit 140 as signal data derived from the photoacoustic wave.

By performing signal processing on a stored digital signal, the computer 150 generates a photoacoustic image. After performing image processing on the obtained photoacoustic image, the computer 150 outputs the photoacoustic image to a display unit 160. The display unit 160 displays an image based on the photoacoustic image. On the basis of a storage instruction from the user or the computer 150, the display image is stored in the storage device 1200 such as a memory in the computer 150 or a data management system connected to a modality via a network.

The computer 150 also performs drive control of the configuration included in the photoacoustic apparatus. The display unit 160 may display a GUI or the like in addition to an image generated by the computer 150. The input section 170 is configured so as to allow the user to input information. Using the input section 170, the user can perform operations such as start and end of measurement and a storage instruction of a generated image.

Details of each component of the photoacoustic apparatus 1100 according to the present embodiment will be described below.

(Light Irradiation Unit 110)

The light irradiation unit 110 includes a light source 111 that emits light and an optical system 112 that guides the light emitted from the light source 111 to the subject 100. The light includes pulsed light such as a so-called square wave or triangle wave.

The pulse width of the light emitted from the light source 111 may be 1 ns or more and 100 ns or less. The wavelength of the light may be in the range of about 400 nm to 1600 nm. When imaging a blood vessel with a high resolution, a wavelength with which absorption in the blood vessel is large (400 nm or more and 700 nm or less) may be used. When imaging a deep portion of a living body, light having a wavelength with which absorption in the background tissue (such as water and fat) of the living body is typically small (700 nm or more and 1100 nm or less) may be used.

As the light source 111, a laser or a light emitting diode can be used. When measurement is performed using light of a plurality of wavelengths, a light source capable of changing the wavelength may be used. When irradiating the subject 100 with light with a plurality of wavelengths, it is also possible to prepare a plurality of light sources that generate light with wavelengths different from one another and to irradiate the subject alternately from the respective light sources. When a plurality of light sources is used, they are collectively expressed as a light source. As the laser, various lasers such as a solid-state laser, a gas laser, a dye laser, and a semiconductor laser can be used. For example, a pulsed laser such as an Nd:YAG laser or an alexandrite laser may be used as a light source. A Ti:sa laser or an optical parametric oscillators (OPO) laser, each using Nd:YAG laser light as excitation light, may be used as a light source. Furthermore, a flash lamp or a light emitting diode may be used as the light source 111. A microwave source may also be used as the light source 111.

As the optical system 112, an optical element such as a lens, a mirror, or an optical fiber can be used. When the breast or the like is used as the subject 100, in order to irradiate the subject with a pulsed light having a wider beam diameter, a light emitting portion of the optical system may be constituted of a diffusing plate or the like that diffuses light. On the other hand, in a photoacoustic microscope, in order to increase the resolution, the light emitting portion of the optical system 112 may be constituted by a lens or the like, and the beam may be focused and emitted.

The light irradiation unit 110 may irradiate the subject 100 with light directly from the light source 111 without including the optical system 112.

(Reception Unit 120)

The reception unit 120 includes a transducer 121 that outputs an electric signal by receiving an acoustic wave and a support 122 that supports the transducer 121. The transducer 121 mayalso be a transmitting unit that transmits an acoustic wave. The transducer as a receiving unit and the transducer as a transmitting unit may be a single (common) transducer or may have different configurations.

As a member constituting the transducer 121, a piezoelectric ceramic material typified by lead zirconate titanate (PLT), a polymer piezoelectric film material typified by polyvinylidene fluoride (PVDF), or the like can be used. Furthermore, an element other than the piezoelectric element may be used. For example, a capacitive transducer (capacitive micro-machined ultrasonic transducers: CMUT), a transducer using a Fabry-Perot interferometer, or the like can be used. Any transducer may be adopted as long as it can output an electric signal by receiving an acoustic wave. The signal obtained by the transducer is a time-resolved signal. That is, the amplitude of the signal obtained by the transducer represents a value based on the sound pressure received by the transducer at each time (e.g., a value proportional to the sound pressure).

The frequency components constituting a photoacoustic wave are typically from 100 KHz to 100 MHz, and a transducer capable of detecting these frequencies may be adopted as the transducer 121.

The support 122 may be made of a metal material having high mechanical strength. In order to make a large amount of irradiation light incident on the subject 100, mirror finishing or light scattering processing may be performed on the surface of the support 122 on the side of the subject 100. In the present embodiment, the support 122 has a hemispherical shell shape and is configured to be capable of supporting the plurality of transducers 121 on the hemispherical shell. In this case, the directional axes of the transducers 121 disposed on the support 122 converge near the curvature center of the hemisphere. When an image is made using signals having been output from the plurality of transducers 121, the image quality near the curvature center becomes high. The support 122 may have any configuration as long as it can support the transducer 121. The support 122 may have a plurality of transducers arranged side by side in a plane or curved surface referred to as a 1D array, 1.5D array, 1.75D array, or 2D array. The plurality of transducers 121 corresponds to a plurality of receiving units.

The support 122 may also function as a container in which an acoustic matching material is stored. That is, the support 122 may be a container for disposing an acoustic matching material between the transducer 121 and the subject 100.

The reception unit 120 may include an amplifier that amplifies a time-series analog signal output from the transducer 121. In addition, the reception unit 120 may include an A/D converter that converts a time-series analog signal output from the transducer 121 into a time-series digital signal. That is, the reception unit 120 may include the signal acquisition unit 140 described later.

In order to be capable of detecting an acoustic wave at various angles, the transducers 121 may be arranged so as to ideally surround the subject 100 from the entire periphery. However, if the subject 100 is too large for the transducers to be arranged so as to surround the entire periphery of the subject 100, the transducers may be arranged on the hemispherical support 122 so as to be close to a state of surrounding the entire periphery. The arrangement and number of the transducers and the shape of the support 122 are only required to be optimized in accordance with the subject 100, and any reception unit 120 can be adopted regarding the present disclosure.

The space between the reception unit 120 and the subject 100 is filled with a medium through which a photoacoustic wave can propagate. For this medium, a material that can propagate an acoustic wave, has acoustic characteristics matching at the interface with the subject 100 and the transducer 121, and has as high a transmittance of the photoacoustic wave as possible is adopted. For example, water, ultrasonic gel, or the like can be adopted as the medium.

FIG. 4 illustrates a side view of the probe 180. The probe 180 according to the present embodiment has the reception unit 120 in which the plurality of transducers 121 is three-dimensionally arranged on the hemispherical support 122 having an opening. The light emitting portion of the optical system 112 is disposed at the bottom of the support 122.

In the present embodiment, as illustrated in FIG. 4, the shape of the subject 100 is held by coming into contact with a holding unit 200.

The space between the reception unit 120 and the holding unit 200 is filled with a medium through which a photoacoustic wave can propagate (acoustic matching material). For this medium, a material that can propagate a photoacoustic wave, has acoustic characteristics matching at the interface with the subject 100 and the transducer 121, and has as high a transmittance of the photoacoustic wave as possible is adopted. For example, water, ultrasonic gel, or the like can be adopted as the medium.

The holding unit 200 as a storage unit is used for holding the shape of the subject 100 during measurement. By holding the subject 100 by the holding unit 200, the movement of the subject 100 can be suppressed and the position of the subject 100 can be kept in the holding unit 200. As the material of the holding unit 200, a resin material such as polycarbonate, polyethylene, or polyethylene terephthalate can be used. That is, it is preferable that the holding unit 200 is made of a material having hardness capable of holding the subject 100. The holding unit 200 may be made of a material that transmits light used for measurement. Furthermore, the holding unit 200 may be made of a material having an impedance like that of the subject 100. In a case where the subject 100 has a curved surface such as a breast, the holding unit 200 may be formed into a recess shape. In this case, the subject 100 can be inserted into the recess portion of the holding unit 200.

The holding unit 200 is attached to an attachment unit 201. The attachment unit 201 may be configured to be capable of replacing a plurality of types of holding units 200 in accordance with the size of the subject. For example, the attachment unit 201 may be configured to be replaceable with a holding unit having a different radius of curvature or a different curvature center.

The holding unit 200 may be provided with a tag in which information of the holding unit 200 is registered and a reading unit that reads the information registered in the tag. For example, information such as the radius of curvature, the curvature center, the sound speed, and the identification ID of the holding unit 200 can be registered in the tag. The information registered in the tag is read out by the reading unit and transferred to the computer 150. In order to easily read the tag when the holding unit 200 is attached to the attachment unit 201, the reading unit may be provided in the attachment unit 201. For example, the tag is a bar code, and the reading unit is a bar code reader.

(Drive Unit 130)

The drive unit 130 is a part that changes the relative position between the subject 100 and the reception unit 120. The drive unit 130 includes a motor such as a stepping motor that generates driving force, a driving mechanism that transmits the driving force, and a position sensor that detects position information of the reception unit 120. As the driving mechanism, a lead screw mechanism, a link mechanism, a gear mechanism, a hydraulic mechanism, or the like can be used. As the position sensor, a potentiometer or the like using an encoder, a variable resistor, a linear scale, a magnetic sensor, an infrared sensor, an ultrasonic sensor, or the like can be used.

The drive unit 130 is not limited to the one that changes the relative position between the subject 100 and the reception unit 120 in the XY direction (two-dimensionally), but the drive unit 130 may change the relative position one-dimensionally or three-dimensionally.

The movement path may be planarly scanned in a spiral manner or in line and space, or tilted along the body surface three-dimensionally. The probe 180 may be moved so as to keep a constant distance from the surface of the subject 100. At this time, the drive unit 130 may measure the moving amount of the probe by monitoring the rotation speed of the motor or the like.

The drive unit 130 may fix the reception unit 120 and move the subject 100 as long as the relative position between the subject 100 and the reception unit 120 can be changed. When the subject 100 is moved, a configuration is conceivable in which the subject 100 is moved by moving the holding unit that holds the subject 100. Both the subject 100 and the reception unit 120 may be moved.

The drive unit 130 may move the relative position continuously or in a step-and-repeat manner. The drive unit 130 may be an electric stage that moves in a programmed trajectory or may be a manual stage.

In the present embodiment, the drive unit 130 performs scanning by simultaneously driving the light irradiation unit 110 and the reception unit 120, but only the light irradiation unit 110 or only the reception unit 120 may be driven.

When the probe 180 is a handheld type provided with a holding portion, the photoacoustic apparatus 1100 may not have the drive unit 130.

(Signal Acquisition Unit 140)

The signal acquisition unit 140 includes an amplifier that amplifies an electric signal that is an analog signal having been output from the transducer 121, and an A/D converter that converts an analog signal having been output from the amplifier into a digital signal. A digital signal having been output from the signal acquisition unit 140 is stored in the computer 150. The signal acquisition unit 140 is also referred to as a data acquisition system (DAS). In the present specification, an electric signal conceptually includes both analog and digital signals. A light detection sensor such as a photodiode may detect light emission from the light irradiation unit 110, and the signal acquisition unit 140 may start the above processing in synchronization with the detection result as a trigger.

(Computer 150)

The computer 150 as an information processing apparatus is constituted by like hardware as the image processing device 1300. That is, the unit carrying the arithmetic function of the computer 150 can be constituted by a processor such as a CPU or a graphics processing unit (GPU) or an arithmetic circuit such as a field programmable gate array (FPGA) chip. These units may be composed of a plurality of processors or arithmetic circuits as fell as a single processor or arithmetic circuit.

The unit carrying the storage function of the computer 150 may be a volatile medium such as a random access memory (RAM). The computer readable storage medium in which a program is stored is a non-temporary computer readable storage medium. The unit carrying the storage function of the computer 150 may be composed of a plurality of computer readable storage media as well as a single computer readable storage medium.

The unit carrying the control function of the computer 150 is constituted by an arithmetic element such as a CPU. The unit carrying the control function of the computer 150 controls the operation each component of the photoacoustic apparatus. The unit carrying the control function of the computer 50 may control each component of the photoacoustic apparatus upon receiving an instruction signal by various operations such as the start of measurement from an input section 170. That is, the computer 150 corresponds to an example of an acceptance unit that accepts an input from the user. The unit carrying the control function of the computer 150 reads out a program code stored in the unit carrying the storage function and controls the operation of each component of the photoacoustic apparatus. That is, the computer 150 can function as a control device of the system according to the present embodiment.

In the present embodiment, the computer 150 includes a one-shot volume data generation unit 151, a reference image generation unit 152, and a moving image generation unit 153 as its functional configuration.

The computer 150 and the image processing device 1300 may be configured by the same hardware. A single piece of hardware may carry the functions of both the computer 150 and the image processing device 1300. That is, the computer 150 may carry the function of the image processing device 1300. The image processing device 1300 may carry the function of the computer 150 as an information processing apparatus.

(One-Shot Volume Data Generation Unit 151)

The one-shot volume data generation unit 151 executes image reconstruction processing using reception signals acquired at positions different from one another for each light irradiation, and generates a plurality of volume data (three-dimensional medical image data). That is, the one-shot volume data generation unit 151 corresponds to the first acquiring unit that acquires photoacoustic image data that is volume data derived from a photoacoustic wave generated by light irradiation to the subject 100. Since this image reconstruction is individually performed for the obtained photoacoustic signal for each light irradiation, the reconstructed photoacoustic image data is referred to as one-shot volume data.

(Reference Image Generation Unit 152)

The reference image generation unit 152 generates reference image data to be referred to for determining moving image conditions. The reference image data is volume data. The reference image generation unit 152 generates reference image data with an improved SN ratio by synthesizing a plurality of one-shot volume data generated by the one-shot volume data generation unit 151. That is, the reference image generation unit 152 corresponds to an example of the second acquiring unit that acquires composition image data by synthesizing at least two or more of the photoacoustic image data.

The moving image conditions are conditions for generating a moving image from a group of one-shot volume data, such as an object region, a maximum intensity projection direction, and the number of one-shot volume data to be used for synthesis when generating two-dimensional image data by rendering processing. The user inputs the determined moving image conditions to the moving image generation unit 153 via the input section 170, for example. The moving image generation unit 153 generates a moving image on the basis of the moving image conditions having been input.

(Moving Image Generation Unit 153)

The moving image generation unit 153 generates a moving image (moving image data) on the basis of moving image conditions having been specified. The moving image generation unit 153 generates composition image data by synthesizing the specified number of one-shot volume data. The smaller the number of one-shot volume data used to generate the composition image data is, the higher the time resolution of the image can be obtained. On the other hand, the larger the number of one-shot volume data used to generate the composition image data is, the higher the SN ratio of the image can be obtained. Furthermore, a moving image suitable for observation can be obtained by specifying the number of syntheses of one-shot volume data with which a desired SN ratio and time resolution can be obtained in accordance with an observation object blood vessel.

Subsequently, the moving image generation unit 153 cuts out the specified region from the composition image data, and generates the maximum intensity projection image data from the specified direction.

When the composition image data includes hair, skin, and the like, if the voxel values of them are larger than those of the observation object blood vessel, the blood vessel is not imaged but the hair, skin, and the like are imaged. If the observation object blood vessel is deep in the living body, a superficial blood vessel having a large voxel value is imaged, and a deep blood vessel cannot be observed in some cases. In such a case, an image suitable for observation can be obtained by specifying an imaging region from the composition image data so that hair, skin, superficial blood vessels, and the like are not included in the imaging region.

Finally, the moving image generation unit 153 generates image data for moving image by arranging the generated highest luminance projection images in time-series order of photographing.

(Display Unit 160)

The display unit 160 displays an image based on the reference image data generated by the reference image generation unit 152 and an image based on the image data for moving image generated by the moving image generation unit 153. By sequentially updating and displaying images based on image data for moving image, they are displayed as a moving image. The moving image generation unit 153 may function as a display control unit that causes the display unit 160 to display an image based on the image data for moving image.

The display unit 160 is a display such as a liquid crystal display, an organic electro luminescence (EL) FED, a glasses-type display, or a head-mounted display. The display unit 160 is a device that displays an image, a numerical value of a specific position, or the like based on subject information or the like obtained by the computer 150. Furthermore, the display unit 160 may display a GUI for operating an image or a device.

The display unit 160 and the display device 1400 may be the same display. That is, a single display may carry the functions of both the display unit 160 and the display device 1400.

(Input Section 170)

As the input section 170, an operation console constituted by a user-operable mouse, keyboard, and the like can be adopted. The display unit 160 may be constituted by a touchscreen, and the display unit 160 may be used as the input section 170.

The input section 170 may be configured so as to be capable of inputting information regarding a position or depth to be observed and the like. As the input method, a numerical value may be input, or the information may be input by operating a slider bar. The image displayed on the display unit 160 may be updated in accordance with the information having been input. This allows the user to set an appropriate parameter while confirming the image generated by the parameter determined by the user's own operation.

The input section 170 and the input device 1500 may be the same device. That is, a single device may carry the functions of both the input section 170 and the input device 1500.

(Subject 100)

Although the subject 100 does not constitute the system, it will be described below. The system according to the present embodiment can be used for the purpose of diagnoses of a malignant tumor, a blood vessel disease, and the like of a human and an animal, follow-up of a chemotherapy, and the like. Therefore, the subject 100 is assumed to be a living body, specifically, diagnosis object sites such as a breast, each organ, a blood vessel network, a head, a neck, an abdomen, and a limb including a finger or a toe, of a human body and an animal. For example, if the human body is a measurement object, oxyhemoglobin or deoxyhemoglobin, or a new blood vessel formed in the vicinity of a blood vessel or a tumor containing a large amount of them may be an object of optical absorber. A plaque on the carotid artery wall may be an object of optical absorber. Melanin, collagen, lipid, and the like contained in the skin and the like may be an object of optical absorber. Furthermore, a phantom imitating a living body may be used as the subject 100.

Each component of the photoacoustic apparatus may be configured as a separate device or as a single, integrated device. At least part of the components of the photoacoustic apparatus may be configured as a single, integrated device.

Each device constituting the system according to the present embodiment may be constituted by different hardware respectively, or all devices may be constituted by a single piece of hardware.

That is, the function of the system according to the present embodiment may be constituted by any hardware.

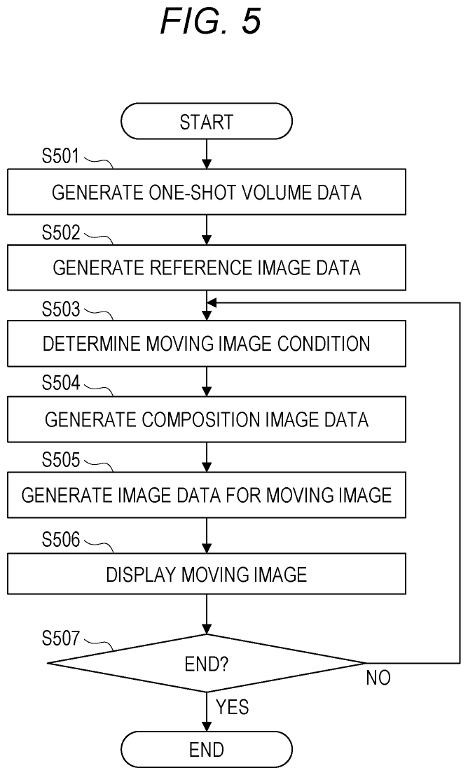

The flow of the image processing method executed by the system according to the present embodiment will be described below. FIG. 5 is a flowchart of the image processing method executed by the system according to the present embodiment. The following photoacoustic apparatus in the present embodiment is mainly used for the purpose of diagnoses of a blood vessel disease, a malignant tumor, and the like of a human and an animal, follow-up of a chemotherapy, and the like. Therefore, part of a living body is assumed as the subject 100.

(Step S501: Generation of One-Shot Volume Data)

In step S501, the photoacoustic apparatus according to the present embodiment acquires a plurality of one-shot volume data by photographing a subject.

Specifically, on the basis of the control parameter specified in step S301, the computer 150 causes the drive unit 130 to move the probe 180 to a specified position. If capturing an image at a plurality of positions is specified in step S310, the drive unit 130 first moves the probe 180 to the first specified position.

Then, the light irradiation unit 110 irradiates the subject 100 and a functional information marker 101 with light on the basis of the control parameter specified in step S310.

The light generated from the light source 111 is emitted to the subject 100 as pulsed light via the optical system 112. Then, the pulsed light is absorbed inside the subject 100, and a photoacoustic wave is generated by the photoacoustic effect. The light irradiation unit 110 transmits a synchronizing signal to the signal acquisition unit 140 together with the transmission of the pulsed light.

Next, upon receiving the synchronizing signal transmitted from the light irradiation unit 110, the signal acquisition unit 140 starts the signal acquisition operation. That is, the signal acquisition unit 140 amplifies and A/D converts the analog electric signal derived from the acoustic wave having been output from the reception unit 120, generates an amplified digital electric signal, and outputs it to the computer 150. The computer 150 stores, in the storage unit, the signal transmitted from the signal acquisition unit 140. The computer 150 may acquire and store the position information of the reception unit 120 at the time of light emission on the basis of the output from the position sensor of the drive unit 130 with light emission as a trigger.

Then, the one-shot volume data generation unit 151, which constitutes the computer 150, executes reconstruction processing by using the signal data stored in the storage unit, and acquires one-shot volume data, and stores them in the storage unit. This reconstruction processing is performed individually for each photoacoustic signal for each light irradiation. At this time, the computer 150 may acquire one-shot volume data on the basis of, in addition to the signal data, control parameters such as the position of the probe 180.

As a reconstruction algorithm that converts the signal data into volume data as a spatial distribution, analytical reconstruction methods such as a back projection method in a time domain and a back projection method in a Fourier domain or a model-based method (iterative operation method) can be adopted. For example, back projection methods in time domain include universal back-projection (UBP), filtered back-projection (FBP), and phased addition (delay-and-sum).

The one-shot volume data generation unit 151 may acquire absorption coefficient distribution information by calculating the light fluence distribution inside the subject 100 of the light emitted to the subject 100 and dividing the initial sound pressure distribution by the light fluence distribution. In this case, the absorption coefficient distribution information may be acquired as photoacoustic image data. The one-shot volume data generation unit 151 can calculate the spatial distribution of the light fluence inside the subject 100 by a method of numerically solving a transport equation or a diffusion equation indicating the behavior of light energy in a medium absorbing and scattering light. As a method of numerically solving, a finite element method, a difference method, a Monte Carlo method, or the like can be adopted. For example, the one-shot volume data generation unit 151 may calculate the spatial distribution of the light fluence inside the subject 100 by solving the light diffusion equation presented in equation (2).

$$\begin{matrix}[ Math . 2 ] & \\ \frac{1}{c} \frac{\partial }{\partial t} Φ ( r , t ) = - {μ}_{a} Φ ( r , t ) + ∇ \cdot ( D ∇ Φ ( r , t ) ) + S ( r , t ) & Equation ( 2 )\end{matrix}$$

Here, D is a diffusion coefficient, μa is an absorption coefficient, S is an incident intensity of the irradiation light, φ is an arriving light fluence, r is a position, and t is a time.

In this step, the one-shot volume data generation unit 151 may acquire the absorption coefficient distribution information corresponding to each of light beams with a plurality of wavelengths. Then, on the basis of the absorption coefficient distribution information corresponding to each of the light beams with the plurality of wavelengths, the one-shot volume data generation unit 151 may acquire, as functional information, the spatial distribution information of the concentration of the substance constituting the subject 100.

The computer 150 as an information processing apparatus that is an apparatus different from the modality may execute the image processing method according to the present embodiment. In this case, the computer 150 acquires the image data generated by the modality in advance by reading out it from the storage unit such as a picture archiving and communication system (PACS), and applies the image processing method according to the present embodiment to the image data. Thus, the image processing method according to the present embodiment can also be applied to previously generated image data.

One-shot volume data generated by the one-shot volume data generation unit 151 is sent to the reference image generation unit 152 and the moving image generation unit 153.

(Step S502: Generation of Reference Image Data)

In step S502, by using the one-shot volume data generated in step S501, the reference image generation unit 152 generates reference image data to be used as a criterion for the user to determine the moving image condition.

The reference image generation unit 152 may combine a plurality of one-shot volume data by adding, averaging, performing an arithmetic-geometric mean, or the like. These pieces of processing generate three-dimensional reference image data in which artifact is suppressed. An image based on the reference image data is displayed on the display unit 160, and an instruction on a moving image condition from the user can be accepted. A plurality of the reference image data may also be generated here, and a plurality of reference image data may be generated in accordance with a pattern when the number of one-shot volume data used for synthesis is changed.

At this time, for example, the reference image generation unit 152 may generate reference image data by selectively synthesizing a volume having a small fluctuation of an observation object blood vessel among a plurality of one-shot volume data. That is, composition image data may be acquired by synthesizing the photoacoustic image data having small displacement amounts of the relative positions between the subject 100 and the probe 180. Specifically, the user specifies a region including the observation object blood vessel from the one-shot volume data. The reference image generation unit 152 compares voxel values (pixel values) of a specified region with respect to the plurality of volume data, and calculates the displacement amounts of the respective volume data. The reference image generation unit 152 acquires a reference image by selectively synthesizing volume data having a small displacement amount. By performing such synthesis, it is possible to obtain reference image data in which blur of the object blood vessel due to position fluctuation during photographing is suppressed.

The reference image data generated by the reference image generation unit 152 is displayed as a reference image on the display unit, and is used to determine a moving image condition.

(Step S503: Determination of Moving Image Condition)

In step S503, the moving image generation unit 153 accepts the instruction from the user and determines a moving image condition on the basis of the reference image displayed on the display unit 160. The moving image conditions to be determined here include, for example, a region of an image from which a moving image is generated, the sight direction (the maximum intensity projection direction in a case of the maximum intensity projection) when converting composition image data into two-dimensional image data, the number of image data to be used for synthesis, or the gradation of the image data to be used for synthesis.

In order to make the two-dimensional image data after rendering that is used as a frame of a moving image after rendering suitable for observation of the object blood vessel, the user may determine, from among the three-dimensional reference image data, a region where rendering processing is provided on the two-dimensional image data. The specification of the region may be carried out by reading a numerical value such as a coordinate value or a voxel number from the reference image, or may be carried out by directly specifying the region on the display screen by a drag operation using an operation console such as a mouse. The imaging region is determined so as not to include, for example, pixels having high pixel values representing hair or skin so that the two-dimensional image after rendering becomes suitable for observation of the object blood vessel. That is, for example, a region that does not include a pixel having a pixel value equal to or greater than a threshold value may be determined as an image region with which the moving image data is generated.

Rendering methods include, for example, the maximum intensity projection (MIP). On the basis of the reference image, the user can obtain a free cross-sectional image by arbitrarily specifying the direction in which the highest value is projected. The user determines the maximum intensity projection direction (MIP direction) in a direction in which observation of the object blood vessel to be observed with a moving image is made easy. The rendering method is not limited to the above and is realized by various known methods.

The number of image data used for synthesis may be determined in consideration of the feature quantity of the observation object region and the SN ratio and the time resolution of the composition image data. In general, the larger the number of volume data used for synthesis is, the more the reduction effect of artifacts and the reduction effect of system noise increases, and a composite volume image with a good image quality can be confirmed, but the time resolution decreases. Therefore, for example, when the observation object region is a thick blood vessel, the thick blood vessel can be observed even if the SN ratio of the composition image data is low, and hence the number of image data to be used for the synthesis is reduced in order to shorten the time required for the synthesis. On the other hand, when the observation object region is a thin blood vessel, an image having a higher SN ratio is required, and hence the number of image data to be used for the synthesis is increased. That is, the thinner the observation object blood vessel is, the larger the number of image data to be synthesized may be. The above is an example, and the number of image data may not necessarily be increased in proportion to the thinness of the blood vessel.

Furthermore, the number of volume data used for the synthesis may be displayed together with the synthesis volume image when the number of the volume data used for the synthesis is changed. This can improve the convenience to the user. In addition, a period (Hz) that can be calculated from the acquisition duration of the photoacoustic signal used for synthesis may be displayed. This helps the time resolution of the obtained image to be easily recognized, and the user can easily determine the number of volume data to be used for synthesis.

The user inputs the determined moving image condition to the moving image generation unit 153 via the input section 170. The series of specification operations performed by the user may be performed by operating the GUI displayed on the display unit 160.

(Step S504: Generation of Composition Image Data)

The moving image generation unit 153 generates composition image data by synthesizing the plurality of one-shot volume data generated in step S501. The moving image generation unit 153 may combine a plurality of one-shot volume data by adding, averaging, performing an arithmetic-geometric mean, or the like. These pieces of processing generate composition image data in which artifact is suppressed. If the volume data is derived from a photoacoustic wave, the moving image generation unit 153 may acquire spectral information by synthesizing a plurality of one-shot volume data corresponding to a plurality of wavelengths generated by light irradiation with wavelengths different from one another. For example, using one-shot volume data indicating the spatial distribution of an absorption coefficient corresponding to light with two wavelengths, the spatial distribution of oxygen saturation is acquired as spectral information. In this case, the moving image generation unit 153 may perform synthesis processing that acquires the oxygen saturation SO₂ according to the equation (1).

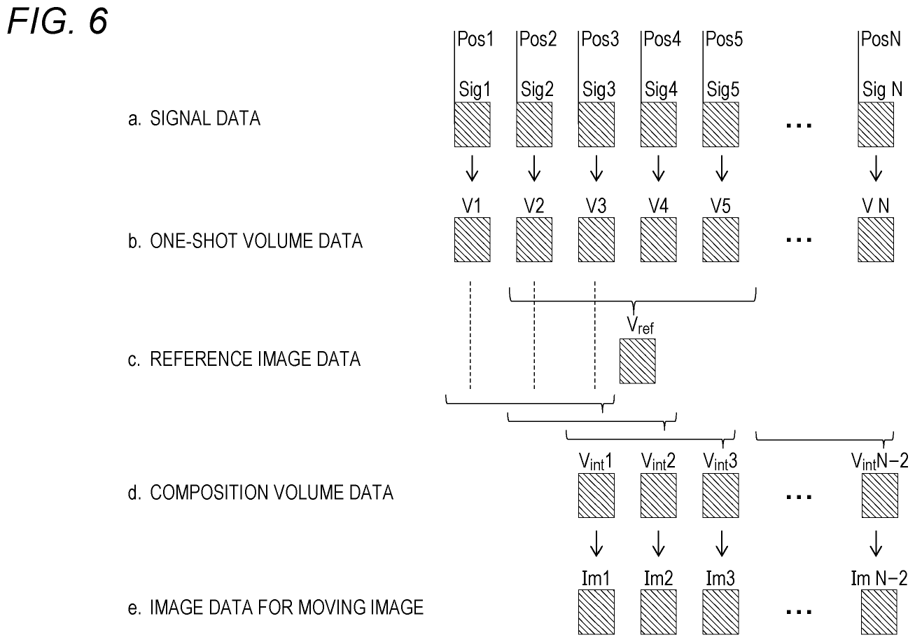

Hereinafter, the relationship between the data processing performed by the signal acquisition unit 140 and the computer 150 will be described with reference to FIG. 6. Photoacoustic signal data acquired when the relative positions of the subject 100 and the probe 180 are Pos1, Pos2, Pos3, and . . . PosN are denoted by Sig1, Sig2, Sig3, and . . . SigN. When the photoacoustic signal is acquired by reception circuit system at each relative position, the one-shot volume data generation unit 151 generates one-shot volume data V1, V2, . . . , and VN by image reconstruction using the acquired photoacoustic signal. Here, the reference image generation unit 152 generates reference image volume data Vref by using a plurality of one-shot volume data. By synthesizing a plurality of one-shot volume data in which the relative positions of the subject 100 and the probe 180 are different from each other, it is possible to generate an image in which artifacts are suppressed. Furthermore, by selectively synthesizing, among the plurality of one-shot volumes, the volume in which the fluctuation of the observation object blood vessel is small, it is possible to obtain reference image data in which blur of the object blood vessel due to the fluctuation during photographing is suppressed. The generated reference image data is displayed on the display unit 160, and the moving image condition is determined by the user.

On the basis of the determined moving image condition, the moving image generation unit 153 generates composition image data Vᵢₙₜ1, Vᵢₙₜ2, and . . . VᵢₙₜN−2 by using a plurality of one-shot volume data. The smaller the number of one-shot volume data used to generate composition image data is, the higher the time resolution of the image is obtained, and on the contrary, the larger the number of one-shot volume data used to generate composition image data is, the higher the SN ratio of the image is obtained. The moving image generation unit 153 acquires, via the input section 170, information regarding the number of syntheses and the combination of volume data to be synthesized, and can change the combination of one-shot volume data used to generate a composition image.

(Step S505: Generation of Image Data for Moving Image)

In step S505, by rendering the three-dimensional composition image data generated in step S504, the moving image generation unit 153 generates two-dimensional image data for moving image corresponding to one frame of the moving image. A plurality of composition image data is rendered to generate a two-dimensional image data group arranged in time series order as image data for moving image. That is, the image processing device 102 generates two-dimensional image data for moving image Im1, Im2, . . . , and ImN−2 from the composition image data Vᵢₙₜ1, Vᵢₙₜ2, . . . , and VᵢₙₜN−2.

As a rendering method, it is possible to adopt any method such as maximum intensity projection (MIP), volume rendering, and surface rendering. Here, the region and the sight direction when rendering three-dimensional composition image data into a two-dimensional image are determined by the moving image condition specified by the user.

To generalize and describe the operations described above, in the present embodiment, a photoacoustic signal is acquired at each of N places (N is an integer equal to or greater than 3) having different relative positions with respect to the subject 100, accordingly acquiring N volume data.

Then, any number of volume data out of the N volume data are synthesized to obtain reference image data. Next, a moving image condition is determined on the basis of the reference image data. Then, on the basis of the determined moving image condition, first composition image data is generated by synthesizing at least two or more volume data of the i-th to (i+m)-th one-shot volume data (i+n<N; both i and m are natural numbers). Furthermore, second composition image data is generated by synthesizing at least two or more volume data of the (i+n)-th to (i+n+m)-th one-shot volume data (n is a natural number). Thereafter, by updating an image based on the first composition image data with an image based on the second composition image data, the first composition image and the second composition image are sequentially displayed.

(Step S506: Display of Moving Image)

The display unit 160 sequentially updates and displays the image data for moving image generated in step S505. The user observes the displayed moving image and determines whether a desired moving image has been obtained. The user confirms the imaging region, the maximum intensity projection direction, the gradation setting, the observation object SN ratio, and the state of time change, and if change is determined to be necessary, the flow of process proceeds to step S503 to reset the moving image generation condition such as the number of one-shot volume data to be used for the composition image data.

(Step S507: End?)

If, as a result of observing the moving image displayed on the display unit 160 in step S507, the user determines that the moving image generation condition needs to be changed, the flow of process proceeds to step S503 to reset the moving image generation condition.

If the user determines that the moving image condition needs to be changed, the user can give an instruction on a change of the moving image condition by using the input section 170. When receiving information of the changed moving image condition via the input section 170, the moving image generation unit 153 can reset the moving image condition. Then, the flow of process returns to step S504 to perform moving image generation again. On the other hand, when there is no instruction to change the moving image condition, the moving image display under the moving image condition having been set is continued.

For example, the moving image generation unit 153 can change, as moving image conditions, the number of one-shot volume data used for composition image data, the rendering conditions (imaging region, maximum intensity projection direction, and gradation setting) of image data for moving image, and the like.

As described above, according to the photoacoustic apparatus according to the present embodiment, a moving image condition can be determined on the basis of a reference image, and a moving image suitable for observation can be generated.

### Second Embodiment

In the first embodiment, the case in which the one-shot volume data used to generate the reference image and the one-shot volume data used to generate the moving image data are the same has been described. However, the one-shot volume data may be different between the data used to generate a reference image and the data used to generate moving image data.

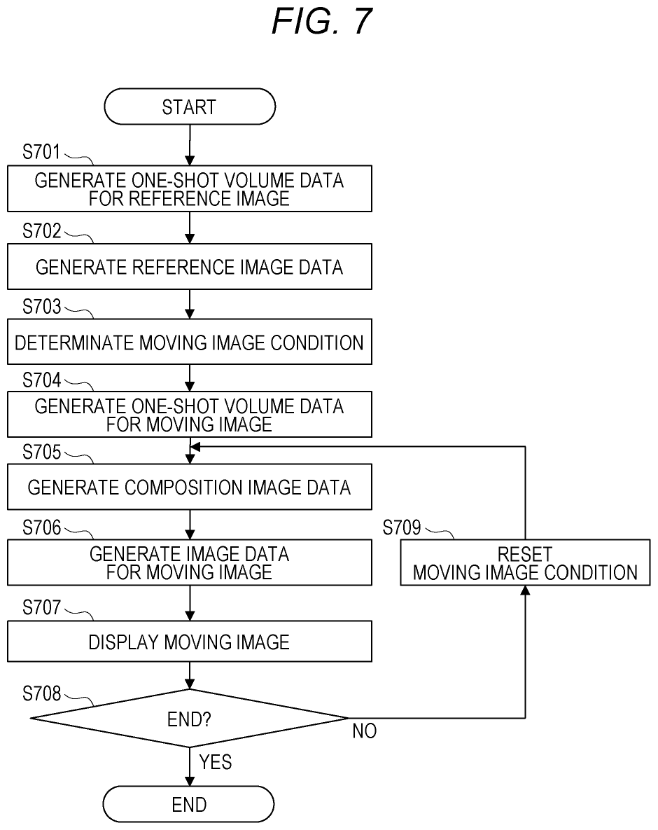

FIG. 7 is a diagram illustrating a flow up to moving image generation in the present embodiment. Here, differences from the first embodiment will be described.

(Step S701: Step of Generating One-Shot Volume Data for Reference Image)

In step S701, the photoacoustic apparatus irradiates the subject 100 with light after the subject 100 is placed in a specified posture. Here, for the purpose of generating a reference image, measurement for the reference image is performed separately from measurement for the moving image.

In order to enhance the time resolution of moving image data, an image of one frame is synthesized with the number of one-shot volumes as few as possible. By making the region of the subject 100 visualized by the moving image data to be about the same size as the high-sensitivity region of the probe 180, the SN ratio can be improved with a small number of syntheses. Since the region to be visualized by the moving image is a relatively narrow region of about the high-sensitivity region of the probe 180, the observation object blood vessel may not be appropriately included the moving image visualizing region.

Here, the reference image is an image in which a wider region than the moving image is visualized. The photoacoustic apparatus acquires subject information for a reference image in a wider range than that at measurement for moving image data. Since the reference image is a still image, there is no constraint on time resolution, and the reference image can be an image of a wide region using one-shot volume data at a multitude of positions. The observation object blood vessel can be easily searched by using a reference image in which a wider region than a moving image is visualized. By specifying the position of the searched observation object blood vessel and measuring the moving image data, the observation object blood vessel can be appropriately included in the moving image region.

The measurement for the reference image may be performed with a wavelength different from that of the measurement for the moving image. For example, a method is conceivable with which by measuring at a shorter wavelength where the optical absorption of melanin is larger than that of the moving image measurement, the skin is visualized more strongly and specification of the moving image region is made easy.

The one-shot volume data generation unit 151 generates one-shot volume data for the reference image and outputs it to the reference image generation unit 152.

(Step S703: Determination of Moving Image Condition)

The user determines the moving image condition on the basis of the reference image displayed on the display unit 160. Here, the moving image condition includes position information for measuring the moving image, in addition to the imaging region, the MIP direction, and the number of one-shot volumes used for a composition image. In the present embodiment, after the position at which the moving image measurement is performed is determined, the subject measurement for generating the moving image data is started.

The user determines the moving image condition with reference to the reference image displayed on the display unit 160. By superimposing and displaying, on the reference image, a frame line indicating the position and size of the region to be made to a moving image, the display unit 160 can improve convenience when the user determines the moving image measurement position. The determined moving image measurement position information is input to the computer 150, and the measurement for moving image is performed.

(Step S704: Generation of One-Shot Volume Data for Moving Image)

The photoacoustic apparatus performs measurement for moving image on the basis of the moving image measurement position information. The one-shot volume data generation unit 151 executes image reconstruction processing using a reception signal of a photoacoustic wave received by the probe 180, and generates one-shot volume data.

(Step S709: Resetting of Moving Image Condition)

If, as a result of observing the moving image displayed on the display unit 160, the user determines in step S708 that the moving image generation condition needs to be changed, the flow of process proceeds to step S709 to reset the moving image generation condition. For example, the moving image generation unit 153 can change, as moving image conditions, the number of one-shot volume data used for composition image data, the rendering conditions (imaging region, maximum intensity projection direction, and gradation setting) of image data for moving image, and the like.

As described above, according to the photoacoustic apparatus according to the present embodiment, a moving image condition can be determined on the basis of a reference image, and a moving image suitable for observation can be generated.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. Fhe storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™) a flash memory device, a memory card, and the like.

While the present disclosure has been described with reference to exemplary embodiments, it is to be understood that the present disclosure is not limited to the particular disclosed exemplary embodiments. The scope of the following claims is to be accorded the broadest interpretation so as to encompass all such modifications and equivalent structures and functions.

This application claims the benefit of Japanese Patent Application No. 2019-064696, filed Mar. 28, 2019, which is hereby incorporated by reference herein in its entirety.

What is claimed is:

1. An image processing device, comprising:
a first acquiring unit configured to acquire photoacoustic image data including volume data derived from a photoacoustic wave generated by light irradiation to a subject;
a second acquiring unit configured to acquire composition image data by synthesizing at least two or more of the photoacoustic image data;
a determination unit configured to determine a generation condition of moving image data based on the composition image data; and
a generation unit configured to generate the moving image data from the composition image data based on the generation condition.

2. The image processing device according to claim 1, wherein the second acquiring unit acquires the composition image data by synthesizing at least two or more of the photoacoustic image data having different relative positions between the subject and a probe.

3. The image processing device according to claim 1, wherein the second acquiring unit generates a plurality of the composition image data from the photoacoustic image data.

4. The image processing device according to claim 1, wherein the second acquiring unit acquires the composition image data by synthesizing at least two or more of the photoacoustic image data having small displacement amounts of the relative positions.

5. The image processing device according to claim 1, wherein the second acquiring unit acquires the composition image data by synthesizing a number of the photoacoustic image data, the number based on a feature amount of an observation object region.

6. The image processing device according to claim 5, wherein the second acquiring unit increases the number of the photoacoustic image data to be synthesized as an observation object blood vessel becomes thinner.

7. The image processing device according to claim 1, wherein the determination unit determines, as the generation condition, at least any one of an image region with which the moving image data is generated, a maximum intensity projection direction, a number of the photoacoustic image data to be synthesized, and gradation of the image data.

8. The image processing device according to claim 1, wherein the determination unit determines a region that does not include a pixel having a pixel value equal to or greater than a threshold value as an image region with which the moving image data is generated.

9. The image processing device according to claim 1, wherein the generation unit generates the moving image data by using a two-dimensional image obtained by rendering processing on the composition image data.

10. The image processing device according to claim 9, wherein the generation unit generates the moving image data by using maximum intensity projection image data obtained by projecting the composition image data with maximum intensity based on the determined maximum intensity projection direction.

11. The image processing device according to claim 1, wherein the generation unit generates the moving image data by using a number of the photoacoustic image data, the number based on thickness of an observation object blood vessel.

12. The image processing device according to claim 1, wherein the second acquiring unit generates the composition image data by synthesizing the photoacoustic image data more in number than the photoacoustic image data used by the generation unit to generate the moving image data.

13. The image processing device according to claim 1, further comprising:
an acceptance unit configured to accept an input from a user, wherein
the determination unit determines the generation condition based on the input.

14. The image processing device according to claim 1, further comprising
a display control unit configured to cause a display unit to display, as an image or a moving image, at least one of the photoacoustic image data, the composition image data, and the moving image data.

15. The image processing device according to claim 14, wherein
the display control unit displays, on the display unit, the composition image data as a reference image, and
the determination unit presents the reference image to a user and determines a generation condition by receiving an input from the user regarding the generation condition of the moving image data.

16. An image processing device, comprising:
a first acquiring unit configured to acquire photoacoustic image data including volume data derived from a photoacoustic wave generated by light irradiation to a subject;
a second acquiring unit configured to acquire composition image data by synthesizing at least two or more of the photoacoustic image data;
a determination unit configured to determine a generation condition of a moving image data based on the composition image data; and
a generation unit configured to generate moving image data by using the photoacoustic image data different from the synthesized photoacoustic image data based on the generation condition.

17. The image processing device according to claim 16, wherein the generation unit generates moving image data by using the photoacoustic image data obtained from light irradiation with a wavelength different from a wavelength of the synthesized photoacoustic image data.

18. An image processing method, comprising:
acquiring photoacoustic image data including volume data derived from a photoacoustic wave generated by light irradiation to a subject;
acquiring composition image data by synthesizing at least vo or more of the photoacoustic image data;
determining a generation condition of a moving image data based on the composition image data; and
generating moving image data from the composition image data based on the generation condition.

19. An image processing method, comprising:
acquiring photoacoustic image data including volume data derived from a photoacoustic wave generated by light irradiation to a subject;
acquiring composition image data by synthesizing at least two or more of the photoacoustic image data;
determining a generation condition of a moving image data based on the composition image data; and
generating moving image data by using the photoacoustic image data different from the synthesized photoacoustic image data based on the generation condition.

20. A non-transitory storage medium that stores a program of instructions that when executed by a computer causes the computer to perform an image processing method including:
acquiring photoacoustic image data including volume data derived from a photoacoustic wave generated by light irradiation to a subject;
acquiring composition image data by synthesizing at least two or more of the photoacoustic image data;
determining a generation condition of a moving image data based on the composition image data; and
generating moving image data from the composition image data based on the generation condition.
